SEQUENCE LISTING (2 sequences)

SEQ ID NO: 1 - protein, 16 aa, synthetic construct
  source 1..16: note = glycosylation site
FQLNVIDNMTNHTMLK

SEQ ID NO: 2 - protein, 15 aa, synthetic construct
  source 1..15: note = glycosylation site
DVVSTGSPGDNVTIR

(19) United States
(12) Patent Application Publication
(10) Pub. No.: US 2024/0401005 A1
(43) Pub. Date: Dec. 5, 2024

(54) Production of Thermoresistant Laccases Using White Rot Fungus Coriolopsis Gallica

(71) Applicant: Université Libre De Bruxelles, Brussels (BE)

(72) Inventors: Sigrid Flahaut, Bruxelles (BE); George Songulashvili, Drogenbos (BE)

(21) Appl. No.: 18/695,489

(22) PCT Filed: Sep. 27, 2022
(86) PCT No.: PCT/EP2022/076805

§ 371 Date: Mar. 26, 2024

(30) Foreign Application Priority Data
Sep. 27, 2021 (EP) 21199193.0

(51) Int. Cl. C12N 9/02; C12N 1/14; C12P 21/00; C12R 1/645

(52) U.S. Cl. CPC C12N 9/0061; C12N 1/14; C12P 21/005; C12R 2001/645; C12Y 110/03002

(57) ABSTRACT

The present invention relates to a laccase enzyme product isolated from a Coriolopsis gallica fungal strain. The laccase enzyme product is characterized in that the laccase enzyme is a thermostable enzyme having a maximum activity at 72° C. and retains at least 50% activity relative to the maximum activity after 20 min incubation at a temperature of 80° C. The invention is also directed to a method for the production of a laccase enzyme product according to the invention, said method encompassing conducting submerged fermentation aerobically of a C. gallica fungal strain in a fermentation medium comprising at least 20 g/L of a carbon source selected from a monosaccharide, a disaccharide, an oligosaccharide, a polysaccharide or any combination thereof; a nitrogen source; and minerals, and to which an inducer of laccase production is added, in a tank bioreactor of at least 200 L that is kept in agitation at between 100 rpm and 220 rpm.

### CROSS REFERENCE TO RELATED APPLICATIONS

This application is a continuation of U.S. patent application Ser. No. 16/757,033, filed Apr. 17, 2020, pending, which is a national phase entry under 35 U.S.C. § 371 of International Patent Application PCT/EP2022/076805, filed Sep. 27, 2022, designating the United States of America and published in English as International Patent Publication WO 2023/046984 on Mar. 30, 2023, which claims the benefit under Article 8 of the Patent Cooperation Treaty to European Patent Application Serial No. 21199193.0, filed Sep. 27, 2021, the entireties of which are hereby incorporated by reference.

### INCORPORATION BY REFERENCE

The ST.26 XML Sequence listing named “10913-10360 US-Sequence Listing.XML”, created on Sep. 26, 2022, and having a size of 4,096 bytes, is hereby incorporated herein by this reference in its entirety.

### TECHNICAL FIELD

The present invention is in the field of microbial fermentation and relates in particular to the production of a laccase enzyme from the white rot fungus Coriolopsis gallica and the thermostable laccase obtained.

### BACKGROUND

Laccases (benzenediol: oxygen oxidoreductases EC: 1.10.3.2) are a diverse group of multi-copper enzymes that oxidize a wide variety of organic and inorganic compounds, including diphenols, polyphenols, substituted phenols, diamines and aromatic amines, with concomitant a reduction of molecular oxygen to water. The structure of the laccase active site includes one type-1 copper atom, one type-2 copper atom and two type-3 copper atoms.

Laccase is an economically important enzyme because of its ability to catalyze various oxidation reactions which are useful in paper and pulp industry, in synthesis of chemicals, in food and beverage industry, bioremediation, biosensors and bio-fuel cells. Economical availability of (purified) laccase is an important factor for usage of laccase in industry.

Most laccases described in literature were isolated from higher fungi, especially white rot fungi belonging to the Basidiomycetes. However, extracellular level of laccase enzyme produced by these fungi is low. Under laboratory conditions, selected fungi are capable of high laccase production through optimisation of fungal fermentation medias and physico-chemical parameters.

Songulashvili et al. (2016 Fungal Biology 120:481-488) described pilot scale Coriolopsis gallica fermentation and downstream modelling of growth, laccase production and substrate consumption. Large amount of C. gallica 1184 laccase was obtained in a pure state in a relatively short time. The produced enzyme was characterized by high temperature stability and robustness against physical treatments.

The high number of possible biotechnological applications of laccases, and the thermostable and robust laccase of C. gallica in particular, and their potential uses in the environmental field, necessitates industrial-scale production of active and stable laccase enzymes.

### SUMMARY OF THE INVENTION

The present inventors optimized and scaled-up culture conditions for laccase production by white rot fungus Coriolopsis gallica allowing high yield laccase production in short time. The produced laccase was found thermostable and robust against downstream processing. Accordingly, the present invention relates to the following aspects and embodiments:

(1) A laccase enzyme product isolated from a Coriolopsis gallica fungal strain, characterized in that the laccase enzyme is a thermostable enzyme having a maximum activity at 72° C. and wherein the laccase enzyme retains at least 50% activity relative to the maximum activity after 20 min incubation at a temperature of 80° C.

(2) The laccase enzyme product according to (1), wherein the laccase enzyme retains at least 15% activity relative to the maximum activity after 20 min incubation at a temperature between 80° C. and 91° C. such as at 91° C.

(3) The laccase enzyme product according to (1) or (2), wherein the Coriolopsis gallica fungal strain is selected from the group consisting of: Coriolopsis gallica 1184, Coriolopsis gallica CBS 547.50 and Coriolopsis gallica CBS 576.88.

(4) The laccase enzyme product according to any one of (1) to (3), wherein the laccase enzyme retains at least 20% activity relative to the maximum activity after 20 min incubation at a temperature between 80° C. and 100° C. such as at 100° C.

(5) The laccase enzyme product according to any one of (1) to (4), wherein the laccase enzyme retains at least 30% activity relative to the maximum activity after 20 min incubation at 91° C.

(6) The laccase enzyme product according to any one of (1) to (5), wherein the laccase enzyme retains at least 10% activity relative to the maximum activity after 3 min incubation at 120° C.

(7) The laccase enzyme product according to any one of (1) to (6), wherein the laccase enzyme is N-glycosylated.

(8) The laccase enzyme product according to any one of (1) to (7), wherein the laccase enzyme is N-glycosylated in at least a glycosylation site of SEQ ID NO:1 (FQLNVIDNMTNHTMLK) and/or, preferably two, a glycosylation site of SEQ ID NO:2 (DVVSTGSPGDNVTIR).

(9) The laccase enzyme product according to (7) or (8), wherein the laccase enzyme has a high mannose type glycosylation (i.e. the laccase enzyme glycoprotein comprises glycans that consist of two N-acetylglucosamines and five to nine mannose residues).

(10). The laccase enzyme product according to any one of (7) to (9), wherein the laccase enzyme has a level of N-glycosylation of between 10% and 20%, preferably of about 15%.

(11) The laccase enzyme product according to any one of (1) to (10), wherein the Coriolopsis gallica fungal strain is Coriolopsis gallica 1184.

(12) A method for the production of a laccase enzyme product according to any one of (1) to (11), said method comprising submerged fermentation of a Coriolopsis gallica fungal strain in a (liquid) fermentation medium in an agitated tank bioreactor of at least 200 L, wherein the fermentation medium comprises a carbon source, wherein said carbon source comprises or consists of a monosaccharide, a disaccharide, an oligosaccharide, a polysaccharide or any combination thereof, preferably a monosaccharide, more preferably glucose; a nitrogen source; and minerals, preferably minerals selected from the group consisting of K, Na, Mn, Mg and Cu, wherein an inducer (or stimulator) of laccase production, such as cupper or manganese ions, or a small-molecule aromatic compound (e.g. tryptophan, tyrosine, vanillin, veratryl alcohol, guaiacol, cinnamic acid, xylidine and lignin), is added to the fermentation medium (to improve laccase production by the microorganism), wherein the fermentation is conducted aerobically, and wherein the bioreactor is kept in agitation at between 100 rpm and 220 rpm, preferably between 120 rpm and 200 rpm.

(13) A method for the production of a laccase enzyme comprising submerged fermentation of a Coriolopsis gallica fungal strain in a (liquid) fermentation medium in an agitated tank bioreactor of at least 200 L,
wherein the fermentation medium comprises a carbon source, wherein said carbon source comprises or consists of a monosaccharide, a disaccharide, an oligosaccharide, a polysaccharide or any combination thereof, preferably a monosaccharide, more preferably glucose; a nitrogen source; and minerals, preferably minerals selected from the group consisting of K, Na, Mn, Mg and Cu,
wherein an inducer (or stimulator) of laccase production, such as cupper or manganese ions, or a small-molecule aromatic compound (e.g. tryptophan, tyrosine, vanillin, veratryl alcohol, guaiacol, cinnamic acid, xylidine and lignin), is added to the fermentation medium (to improve laccase production by the microorganism),
wherein the fermentation is conducted aerobically, and
wherein the bioreactor is kept in agitation at between 100 rpm and 220 rpm, preferably between 120 rpm and 200 rpm.

(14) The method according to (12) or (13), wherein the Coriolopsis gallica strain is selected from the group consisting of: Coriolopsis gallica 1184, Coriolopsis gallica CBS 547.50 and Coriolopsis gallica CBS 576.88, preferably Coriolopsis gallica 1184.

(15) The method according to any one of (12) tor (14), wherein the monosaccharide, the disaccharide, the oligosaccharide and/or the polysaccharide is not bound to a non-carbohydrate substance such as lignin.

(16) The method according to any one of (12) to (15), wherein the fermentation medium comprises at least 20 g/L or at least 30 g/L, preferably at least 40 g/L, such as between 40 g/L and 60 g/L (e.g. about 50 g/L) of the monosaccharide, the disaccharide, the oligosaccharide and/or the polysaccharide carbon source.

(17) The method according to any one of (12) to (16), wherein the nitrogen source is peptone such as bacto™peptone and wherein the carbon source is a monosaccharide, preferably glucose.

(18) The method according to any one of (12) to (17), wherein the fermentation medium comprises 10 to 25 g/L, preferably 17 g/L, peptone such as bacto™peptone; 40 to 60 g/L, preferably 50 g/L, glucose; 0.5 to 5.0 g/L, preferably 2.5 g/L, KH₂PO₄; 0.01 to 0.10 g/L, preferably 0.05 g/L, MnSO₄xH₂O; 0.1 to 1.0 g/L, preferably 0.5 g/L, MgSO₄x7H₂O; and 0.005 to 0.05 g/L, preferably 0.02 g/L, CuSO₄x5H₂O.

(19) The method according to any one of (12) to (18), wherein the inducer of laccase production is vanillin.

(20) The method according to any one of (12) to (19), wherein between 100 μM and 500 μM, preferably 200 M, vanillin is added to the fermentation medium.

(21) The method according to any one of (12) to (20), wherein agitation speed is increased with fungal biomass during fermentation.

(22) The method according to any one of (12) to (21), wherein the temperature during the fermentation is in the range from 25° C. to 32° C., preferably in the range from 27° C. to 30° C., more preferably about 30° C.

(23) The method according to any one (12) to (22), wherein the level of dissolved oxygen is at least 25% of the saturation amount, preferably between 25% and 35% of the saturation amount.

(24) The method according to any one of (12) to (23), wherein the pH during the fermentation is in the range from 3.0 to 7.0, preferably from 3.5 to 6.0, more preferably from 3.8 to 5.5.

(25) The method according to any one of (12) to (24), wherein the fermentation period is less than 10 days, preferably between 6 and 8 days, more preferably 7 days.

(26) The method according to any one of (12) to (25), further comprising the steps of growing said Coriolopsis gallica fungal strain in an inoculum medium to obtain an inoculum, and adding said inoculum to the fermentation medium.

(27) The method according to (26), wherein the inoculum medium comprises glucose, peptone, yeast extract, and minerals.

(28) The method according to any one of (12) to (27), further comprising the step of recovering the laccase from the fermentation medium.

(29) The method according to claim 28), wherein said recovery comprises biomass separation and optionally one or more concentration steps resulting in a concentrated laccase solution.

(30) The method according to (29), wherein the concentration steps comprise a microfiltration step, an ultrafiltration step and a diafiltration step.

(31) The method according to (29) or (30), wherein the concentrated laccase solution is lyophilized or atomized.

(32) A laccase enzyme product obtainable by the method according to any one of (13) to (31).

(33) The laccase enzyme product according to any one of (1) to (11), which is obtainable by the method according to any one of (12) to (31).

### BRIEF DESCRIPTION OF THE FIGURES

The teaching of the application is illustrated by the following Figures which are to be considered as illustrative only and do not in any way limit the scope of the claims.

FIG. 1 provides an overview of a 200 L scale process for producing laccase from C. gallica according to an embodiment of the invention.

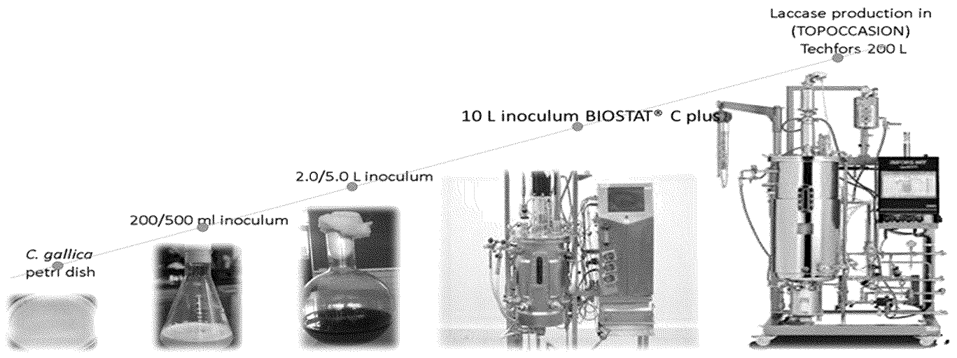

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In an t aspect, the present invention relates to a method for the production of a laccase enzyme product as described herein comprising submerged fermentation of a Coriolopsis gallica fungal strain in a liquid fermentation medium in an agitated tank bioreactor of at least 200 L,
wherein the fermentation medium comprises at least 20 g/L, preferably at least 40 g/L of a monosaccharide carbon source, preferably glucose; a nitrogen source; and minerals, preferably minerals selected from the group consisting of K, Na, Mn, Mg and Cu,
wherein an inducer of laccase production is added to the fermentation medium,
wherein the fermentation is conducted aerobically, and
wherein the bioreactor is kept in agitation at between 100 and 220 rpm, preferably between 120 and 200 rpm.

The fermentation process of the invention is performed on an industrial scale. An industrial scale process is understood to encompass a fermentation process on a fermenter volume scale which is at least 100 L, preferably at least 150 L, more preferably at least 200 L.

A “fermenter” as used herein refers to any apparatus suitable for the industrial production of bacterial cultures. However, as used herein, this term does not include culture flasks which are typically used for growth of bacteria on a smaller scale.

The process of the invention can be used with any strain of Coriolopsis gallica. Non-limiting examples of C. gallica strains include Coriolopsis gallica 1184 (Songulashvili et al. 2016), Coriolopsis gallica CBS 547.50 (CBS Filamentous Fungi Collection, Utrecht, The Netherlands) and Coriolopsis gallica CBS 576.88 (CBS Filamentous Fungi Collection, Utrecht, The Netherlands). In embodiments, a C. gallica strain selected from the group consisting of Coriolopsis gallica strain 1184, Coriolopsis gallica strain CBS 547.50 and Coriolopsis gallica strain CBS 576.88 is used. In particular embodiments, the following strain of Coriolopsis gallica is used in the processes of the invention: C. gallica 1184.

In the methods described herein, “submerged fermentation” of a Coriolopsis gallica fungal strain is used. In short, this culture method involves dissolving in water the compounds of the fermentation medium, transferring the solution to a bioreactor and inoculating the bioreactor with fungal biomass. The fungal biomass may be in the form of single hyphae, spores, aggregates of mycelium, and partly differentiated mycelium. The technology related to submerged fermentation of microbial organisms such as fungi is well known for the skilled person.

Submerged fermentation may be conducted as a batch, fed-batch or continuous process. In a batch process, all the necessary materials, with the exception of oxygen for aerobic processes, are placed in a reactor at the start of the operation and the fermentation is allowed to proceed until completion, at which point the product is harvested. In a fed-batch process, the culture is fed continuously or sequentially with one or more media components with the removal of the culture fluid. In a continuous process, fresh medium is supplied and culture fluid is removed continuously at volumetrically equal rates to maintain the culture at a steady growth rate. In preferred embodiments, the method is conducted as a batch process or a fed-batch process.

The production of laccase can be improved by optimizing the culture conditions, as well as the fermentation medium of a Coriolopsis gallica fungal strain. When optimizing the fermentation medium, for example, the effect of the quality (e.g. an organic or inorganic source of nitrogen) and the quantity of the source of nitrogen on the laccase production may be established. Similarly, the effect of the source of carbon on the laccase production may be established. Also the carbon/nitrogen ratio may be optimized for laccase production.

The fermentation medium supports fungal growth and stimulates the production of laccase. The fermentation medium may contain a carbon source, a nitrogen source as well as additional compounds required for growth of the fungal strain and/or the formation of the laccase.

Non-limiting examples of suitable carbon sources known in the art include glucose, wheat bran, maltose, maltodextrins, sucrose, hydrolysed starch, starch, molasses, oils, and combinations thereof. In preferred embodiments, the carbon source comprises or consists of a monosaccharide, a disaccharide, an oligosaccharide, a polysaccharide, or any combination thereof, preferably a monosaccharide. In more preferred embodiments, the carbon source comprises or consists of glucose. Preferably, the disaccharide, the oligosaccharide and/or the polysaccharide are easily hydrolysable, e.g. not bound to lignin, a protein or another non-carbohydrate substance.

The term “carbohydrate” or “saccharide” as used herein refers to molecules consisting of carbon (C), hydrogen (H) and oxygen (O) atoms, usually with a hydrogen-oxygen atom ratio of 2:1 (as in water) and with the empirical formula Cm (H₂O), (where m may or may not be different from n) and includes monosaccharides, oligosaccharides, polysaccharides, and mixtures of monosaccharides, oligosaccharides and/or polysaccharides. The term “monosaccharide” as used herein refers to the simplest carbohydrates in that they cannot be hydrolyzed to smaller carbohydrates. They are the most basic units of carbohydrates and have the general chemical formula Cm (H₂O) n (where m may or may not be different from n). The term “disaccharide” as used herein refers to a carbohydrate substance composed of two monosaccharide molecules that are joined by a glycosidic linkage. The term “oligosaccharide” refers to a polymeric carbohydrate containing typically three to ten monosaccharide molecules that are joined via glycosidic bonds. The term “polysaccharide” as used herein means a polymeric carbohydrate molecule composed of long chains of monosaccharide units (typically more than 10 monosaccharides) bound together by glycosidic linkages and on hydrolysis give the constituent monosaccharides, disaccharides and/or oligosaccharides. Exemplary monosaccharides include, without limitation, C6 sugars (e.g., fructose, mannose, galactose, or glucose) and C5 sugars (e.g., xylose or arabinose). Non-limiting examples of disaccharides include sucrose, lactose, maltose, trehalose, cellobiose. Exemplary oligosaccharides include, without limitation, dextran or glucan. Exemplary polysaccharides include, without limitation, starch and cellulose.

Non-limiting examples of nitrogen sources known in the art include peptone, bacto™peptone, soy bean meal, corn steep liquor, yeast extract, ammonia, ammonium salts, nitrate salts, urea. In embodiments, the nitrogen source is selected from peptone, bacto™peptone, yeast extract, or a combination thereof, preferably peptone or bacto™peptone.

Non-limiting examples of additional compounds include phosphate; sulphate; salts providing magnesium, sodium, manganese, copper, and potassium; trace elements and/or vitamins.

The total amount of carbon and nitrogen source in a fermentation medium may vary depending on e.g. the needs of the microorganism and/or the length of the fermentation process. The ratio between carbon and nitrogen source in a fermentation medium may vary considerably, whereby one determinant for an optimal ratio between carbon and nitrogen source is the elemental composition of the product to be formed.

Additional compounds required for growth of a microorganism and/or for product formation, like phosphate, sulphate or trace elements, may be added in amounts that may vary depending on the microorganism and the type of product that is formed. Typically, the amount of medium components necessary for growth of a microorganism may be determined in relation to the amount of carbon source used in the fermentation medium, since the amount of biomass formed will be primarily determined by the amount of carbon source used.

The inventors developed synthetic and lignocellulosic media suitable for Coriolopsis gallica laccase production and secretion into the fermentation medium. The developed media also allow high level of fungal biomass production. For example, the synthetic fermentation medium may comprise:
Peptone such as bacto™peptone 10 to 25 g/L, preferably 17 g/L;
Glucose 40 to 60 g/L, preferably 50 g/L;
KH₂PO₄ 0.5 to 5.0 g/L, preferably 2.5 g/L;
MnSO₄xH₂O 0.01 to 0.10 g/L, preferably 0.05 g/L;
MgSO₄x7H₂O 0.1 to 1.0 g/L, preferably 0.5 g/L; and
CuSO₄x5H₂O 0.005 to 0.05 g/L, preferably 0.02 g/L.

The lignocellulosic fermentation medium may comprise:
Peptone 1 to 5 g/L, preferably 2 g/L;
Yeast extract 1 to 5 g/L, preferably 2 g/L;
Glucose 2 to 10 g/L, preferably 5.5 g/L;
Wheat bran 30 to 50 g/L, preferably 40 g/L;
KH₂PO₄ 0.2 to 2 g/L, preferably 0.8 g/L;
Na₂HPO₄x2H₂O 0.05 to 0.5 g/L, preferably 0.25 g/L;
MgSO₄x7H₂O 0.1 to 1.0 g/L, preferably 0.45 g/L; and
CuSO₄x5H₂O 0.05 to 0.5 g/L, preferably 0.25 g/L.

Without wishing to be bound by any theory, glycosylation level and thermostability of the laccase may be improved in fermentation media comprising a monosaccharide, a disaccharide, an oligosaccharide, a polysaccharide, or any combination thereof, in particular a monosaccharide, more particularly glucose. In embodiments, the fermentation medium comprises at least 10 g/L, preferably at least 20 g/L, more preferably at least 30 g/L, even more preferably at least 40 g/L such as between 40 g/L and 60 g/L, most preferably about 50 g/L of a monosaccharide, a disaccharide, an oligosaccharide, a polysaccharide, or any combination thereof, preferably a monosaccharide, more preferably glucose. In further particular embodiments, the fermentation medium comprises peptone such as bacto™peptone; at least 10 g/L, preferably at least 20 g/L, more preferably at least 30 g/L, even more preferably at least 40 g/L such as between 40 g/L and 60 g/L, most preferably about 50 g/L of a monosaccharide, a disaccharide, an oligosaccharide, a polysaccharide, or any combination thereof, preferably a monosaccharide, more preferably glucose; and salts providing minerals. In yet further particular embodiments, the fermentation medium comprises:

| | |
|---|---|
| Peptone such as bacto ™peptone | 10 to 25 g/L, preferably 17 g/L; |
| Glucose | 40 to 60 g/L, preferably 50 g/L; |
| KH₂PO₄ | 0.5 to 5.0 g/L, preferably 2.5 g/L; |
| MnSO₄ × H₂O | 0.01 to 0.10 g/L, preferably 0.05 g/L; |
| MgSO₄ × 7H₂O | 0.1 to 1.0 g/L, preferably 0.5 g/L; and |
| CuSO₄ × 5H₂O | 0.005 to 0.05 g/L, preferably 0.02 g/L. |

To trigger the production of laccase, an “inducer” or “inductor” is added to the fermentation medium. An “inducer” or “inductor”, which terms are used interchangeably herein, refer herein to a compound or composition for improving the capacity of a microorganism strain for producing a laccase. Copper ions is one of the compositions of laccase activity center and an effective stimulator for laccase synthesis. Also some small-molecule aromatic compounds including guaiacol, veratryl alcohol, vanillin and cinnamic acid are used as stimulators for improving laccase production because of the structure similarity to the lignin. Non-limiting examples of laccase inductors that can be used in the methods described herein are copper and manganese ions, aromatic amino acids such as tryptophan and tyrosine, vanillin, veratryl alcohol, guaiacol, cinnamic acid, xylidine and lignin. In preferred embodiments, the inducer is vanillin. Vanillin is a natural inductor of laccase production, and advantageously, not toxic for humans and animals, which allows the laccase, wherein trace amounts of vanillin may be present, to be used in agro-alimentary applications.

The way and the time of adding the inducers, as well as their concentration can be optimised as known to the skilled person. In embodiments, vanillin is added to the fermentation medium at a concentration ranging between about 100 and about 500 μM, preferably at a concentration of about 200 μM.

Another nutritional requirement of Coriolopsis gallica for the production of laccase is oxygen Accordingly, the method of the invention is performed aerobically, i.e. in the presence of oxygen. Oxygen may be fed as air to the liquid fermentation medium. However, it is also possible to feed pure oxygen to the fermentation medium and/or air enriched with oxygen and/or air and oxygen in separate feeds. In preferred embodiments, the level of dissolved oxygen is at least 25% of the saturation amount (i.e., the maximum amount that can be dissolved in the fermentation medium under the conditions of temperature and pressure that are used), preferably between 25% and 35% of the saturation amount. The level of dissolved oxygen may be controlled by known methods. For example, dissolved oxygen may be measured using an oxygen electrode with a gas-permeable membrane (Clark electrode). Also measures that can be taken to modulate the dissolved oxygen levels are commonly known to the skilled person and may include, for example, modulation of at least one of the following parameters: modulating agitation (e.g. speed of the stirrer), modulating aeration, modulating oxygen percentage in the entering gas flow (oxygen enrichment), etc. For example, oxygen may be introduced into the fermentation culture by bubbling compressed air through the culture. Where different concentrations of oxygen are present in the air introduced into the culture, the flow rate should be adapted to take account of this. For instance, where a supply of 100% oxygen is introduced into the culture, the flow rate would be correspondingly lower. Where gas containing less oxygen than air is introduced into the culture, a higher flow rate could be applied.

During submerged fermentation the fermentation medium with the fungal biomass is agitated under conditions sufficient to maintain a homogenous culture, e.g. to reduce the occurrence of gradients and to ensure oxygen availability to the submerged cells. Agitation may be by stirring the culture in the fermenter, or by any other suitable means, for example by agitation using a Rushton turbine and/or gas bubbling. For example, the bioreactor may be agitated by a Rushton turbine. In embodiments, the agitation speed is between 100 and 220 rpm, more preferably between 120 and 200 rpm. The bioreactor may be kept in constant agitation, or agitation speed may be varied, e.g. depending on fungal biomass.

Preferably, the pH of the fermentation medium is adjusted to from about 4.0 to about 7.0, preferably to from about 5.0 to about 6.0, or any pH therebetween, for example to a pH of about 5.2 before the fermentation medium is inoculated with fungal biomass. After the initial adjustment, pH may be dropped naturally during the course of the fermentation, e.g. to a value from about 3.0 to about 5.0, preferably to from about 3.5 to about 4.5, such as to about 4.0, or controlled at a particular value using addition of suitable pH-control agents, such as acid and base. In embodiments, the pH during the fermentation is in the range from 3.0 to 7.0, preferably from 3.5 to 6.0, more preferably from 3.8 to 5.5.

The fermentation takes place at a temperature suitable for the culture of Coriolopsis gallica. In embodiments, the temperature during the fermentation is in the range from 28° C. to 32° C., preferably in the range from 29° C. to 31° C., more preferably about 30° C.

As used herein, the fermentation step or production phase refers to the step in which C. gallica is cultured within the fermenter to produce and secrete laccase. The fermentation step commences with the introduction of the inoculum into the fermenter. The fermentation according to the method of the invention is preferably carried out over a period of less than 10 days, preferably a period of between 3 and 10 days, more preferably a period of between 6 and 8 days such as about 7 days.

In the methods described herein, a production phase wherein the laccase is produced by fungal biomass may be preceded by a growth phase wherein the fungal biomass is formed. Accordingly, in embodiments, the method further comprises the steps of growing said Coriolopsis gallica fungal strain in an inoculum medium to obtain an inoculum, and adding said inoculum to the fermentation medium for submerged fermentation.

For inoculation of the fermentation medium, Coriolopsis gallica mycelium from an inoculum medium in e.g. a shake flask or a bioreactor can be used as an inoculum.

Inoculum medium for Coriolopsis gallica can initially be inoculated with a Coriolopsis gallica mycelium that is maintained on agar plates. Agar plates containing malt extract, e.g. 2% (w/v) malt agar plates, can be used for maintaining Coriolopsis gallica fungal strains. The plates may be inoculated with mycelium from a Coriolopsis gallica strain, e.g. Coriolopsis gallica 1184, and are preferably incubated at 4° C.

The mycelium may be scraped off the malt agar plates and transferred aseptically to inoculum medium in e.g. a shake flask; or malt agar plugs can be used for inoculating the inoculum medium.

The inoculum medium may contain sterile water comprising dissolved nutrient compounds supporting the growth of the fungal mycelium. In particular embodiments, the inoculum medium comprises glucose, peptone such as bacto™peptone, yeast extract, and salts providing minerals, more particularly KH₂PO₄, Na₂HPO₄ and MgSO₄x7H₂O, more particularly, the inoculum medium comprises 5 to 20 g/L, preferably 10 g/L glucose; 1.0 to 5.0 g/L, preferably 2.0 g/L yeast extract; 1.0 to 5.0 g/L, preferably 2.0 g/L peptone such as bacto™peptone; 0.2 to 2.0 g/L, preferably 0.8 g/L KH₂PO₄; 0.05 to 0.5 g/L, preferably 0.2 g/L Na₂HPO₄; and 0.1 to 1.0 g/L, preferably 0.5 g/L MgSO₄x7H₂O. Growth of Coriolopsis gallica may be achieved by incubating the inoculum medium under agitation, e.g. at 120 rpm, for a period of one to several days at a temperature optimal for growth of the fungal cells, preferably at a temperature at 30° C.

By successively inoculating increasing volumes of inoculum medium, submerged cultures of Coriolopsis gallica mycelium can be obtained which are suitable for use in the fermenter. Stationary vat or tank bioreactors equipped with suitable agitation and optionally aeration devices can be used for larger volumes, but for smaller volumes small flasks can be used which are either shaken or stirred by suitable mechanical means.

For example, in particular embodiments, an inoculum is obtained by the successive step of:
inoculating malt agar plugs from a solid (maintenance) culture in 0.2 L inoculum medium in a flask, such as a 0.5 L flask;
incubating the flask on a rotary shaker at 120 rpm and 30° C. for 4 days;
harvesting mycelial pellets from the 0.2 L inoculum medium;
homogenizing the harvested mycelial pellets;
inoculating 200 ml of mycelial homogenate in 2 L inoculum medium in a flask, such as a 5 L flask;
incubating the flask on a rotary shaker at 120 rpm and 30° C. for 5 days;
harvesting the 2 L inoculum medium containing fungal biomass from the flask;
inoculating all the fungal biomass from the flask in a bioreactor (e.g. BIOSTAT® Cplus) containing 10 L inoculum medium;
incubating the bioreactor under stirring at 120 rpm and at 30° C. and at a level of dissolved oxygen of 25% of the saturation amount for 3 days;
harvesting 10 L inoculum medium as inoculum for inoculating the fermenter.

The laccase produced during fermentation is excreted into the liquid fermentation medium. Following fermentation, the fermentation medium containing the laccase enzyme may be used directly, or the laccase enzyme may be recovered from the fermentation medium. The purpose of the recovery process is in one aspect to separate the biomass, purify, concentrate, and stabilize the produced laccase. In embodiments, the method described herein comprise an additional step of recovering the laccase from the fermentation medium.

The extracellular fraction of the fermentation medium is also termed the supernatant and this fraction can be separated from the biomass, including fungal mycelium and optionally “left-overs” of e.g. wheat bran, by conventional processes such as centrifugation, filtration, or by any other means available for obtaining a liquid fraction essentially without any fungal mycelium present therein.

After the biomass has been separated, the supernatant of laccase product may be concentrated by conventional techniques such as microfiltration, ultrafiltration, diafiltration, evaporation or any combination thereof. By microfiltration and ultrafiltration, the supernatant of the enzyme product is separated into a concentrated supernatant and a permeate using a membrane; the permeate is mainly pure water. In ultrafiltration (cut-off of between 2 kDa and 100 kDa, preferably between 2 kDa and 20 kDa, more preferably 10 kDa), the membrane pore size is smaller than in microfiltration (membrane pore size of between 0.2 to 2 μm, preferably 0.4 to 1 μm, more preferably 0.8 μm). Diafiltration is a process wherein addition of water or a salt-containing aqueous solution is effected, in continuous or discontinuous manner, into an ultrafiltration retentate. Simultaneously or subsequently, an equivalent amount of permeate is removed. The result of such operation is to deplete the retentate of filterable elements.

In order to increase stability/storageability of the laccase, the concentrated laccase solution may be lyophilized or atomized. For atomization a carrier may be needed such as maltodextrins or KCl.

A further aspect of the present invention relates to a laccase enzyme product isolated from a Coriolopsis gallica fungal strain, which is obtainable by the method of the invention. The laccase enzyme is characterized as being thermostable. The laccase enzyme has a maximum activity at 72° C. and retains at least 20% activity relative to the maximum activity after 20 min incubation at a temperature between 72° C. and 80° C. such as at 80° C., preferably between 72° C. and 91° C. such as at 91° C., more preferably between 72° C. and 100° C. such as at 100° C. In particular embodiments, the laccase enzyme retains at least 50% activity relative to the maximum activity after 20 min incubation at a temperature of 80° C. In particular embodiments, the laccase enzyme retains at least 15%, preferably at least 20%, more preferably at least 30%, activity relative to the maximum activity after 20 min incubation at 91° C. In particular embodiments, the laccase enzyme retains at least 20% activity relative to the maximum activity after 20 min incubation at 100° C. In particular embodiments, the laccase enzyme retains at least 15% activity relative to the maximum activity after 20 min incubation at a temperature between 72° C. and 91° C. such as at a temperature between 80° C. and 91° C. or at 91° C.

In further embodiments, the laccase enzyme retains at least 60% activity relative to the maximum activity after 3 min incubation at temperatures between 72° C. and 80° C. such as at 80° C., preferably between 72° C. and 91° C. such as at 91° C., more preferably between 72° C. and 100° C. such as at 100° C. In particular embodiments, the laccase enzyme retains maximum activity after 3 min incubation at 80° C. In particular embodiments, the laccase enzyme retains at least 30%, preferably at least 60%, more preferably at least 70%, activity relative to the maximum activity after 3 min incubation at 91° C. In particular embodiments, the laccase enzyme retains at least 60% activity relative to the maximum activity after 3 min incubation at 100° C.

In yet further embodiments, the laccase enzyme retains at least 30% activity relative to the maximum activity after 10 min incubation at temperatures between 72° C. and 80° C. such as at 80° C., preferably between 72° C. and 91° C. such as at 91° C., more preferably between 72° C. and 100° C. such as at 100° C. In particular embodiments, the laccase enzyme retains at least 65% activity relative to the maximum activity after 10 min incubation at 80° C. In particular embodiments, the laccase enzyme retains at least 45% activity relative to the maximum activity after 10 min incubation at 91° C. In particular embodiments, the laccase enzyme retains at least 35% activity relative to the maximum activity after 10 min incubation at 100° C.

In yet further embodiments, the laccase enzyme retains at least 10% activity relative to the maximum activity after 3 min incubation at 120° C.

For determining optimum temperature of a laccase enzyme, reaction mixtures comprising purified laccase enzyme can be incubated at temperatures ranging from e.g. 10° C. to 80° C. with an interval of e.g. 5° C. or 10° C., and determining laccase activity at the end of the incubation as known to the skilled person.

Thermostability of a laccase enzyme can be determined by incubating the laccase enzyme, preferably in a dried form, at various temperatures ranging from e.g. 70° C. to 120° C., each for various periods ranging from 2 to 20 min, e.g. for 3, 10 and 20 min, and determining residual laccase activity at the end of the incubation. In particular embodiments, thermostability is determined according to the method used in the examples.

Laccase activity can be determined by methods well known to the skilled person using e.g. 2,2′-azinobis [3-ethylbenzthiazoline-6-sulfonate] (ABTS), syringaldazine or dimethoxyphenol (DMP) as substrate. The oxidized product of these substrates absorbs in the visible wavelength range and can be easily measured using a spectrophotometer.

The laccase enzyme of the invention may be further characterized as being glycosylated, in particular the laccase enzyme may have N-glycosylation.

As used herein, the term “N-glycan” refers to a N-linked oligosaccharide, e.g., one that is attached by an asparagine-N-acetylglucosamine linkage to an asparagine residue of a polypeptide. N-glycans have a common pentasaccharide core of Man3GlcNAC₂ (“Man” refers to mannose; “Glc” refers to glucose; and “NAc” refers to N-acetyl; GlcNAc refers to N-acetylglucosamine). N-glycans differ with respect to the number and type of sugars or branches (antennae) comprising peripheral sugars (e.g., GlcNAc, fucose, and sialic acid) that are attached to the core structure. In embodiments, the laccase enzyme of the invention comprises N-glycans of the high mannose type. As used herein, a “high mannose” type N-glycan refers to a N-glycan that has five to nine mannose residues.

The glycosylation site refers to the amino acid sequence of the glycosylated polypeptide, in particular the glycosylated laccase, to which a N-glycan is attached. In embodiments, the laccase enzyme of the invention enzyme is N-glycosylated in at least one glycosylation site selected from the group consisting of: FOLNVIDNMTNHTMLK (SEQ ID NO:1) and DVVSTGSPGDNVTIR (SEQ ID NO:2), preferably in at least the glycosylation site of SEQ ID NO:1 and the glycosylation site of SEQ ID NO:2.

As used herein, the degree or level of glycosylation refers to the account of carbohydrates on total mass of the laccase. The degree or level of glycosylation can be determined based on the change, in particular the decrease, in molecular weight before and after deglycosylation analysis as known to the skilled person. For deglycosylation analysis, Peptide-N-Glycosidase F (PNGase F) may be used. In embodiments, the laccase enzyme of the invention has a level of N-glycosylation of between 10% and 20%, preferably of about 15%. In embodiments, deglycosylation of the laccase may result in a decrease of molecular weight of 9.5 kDa.

Certain aspects and embodiments of the present invention are set forth in the below numbered statements:

(1) A method for the production of a laccase enzyme comprising submerged fermentation of a Coriolopsis gallica fungal strain in a (liquid) fermentation medium in an agitated tank bioreactor of at least 200 L,
wherein the fermentation medium comprises a carbon source, wherein said carbon source comprises or consists of a monosaccharide, a disaccharide, an oligosaccharide, a polysaccharide or any combination thereof, preferably a monosaccharide, more preferably glucose; a nitrogen source; and minerals, preferably minerals selected from the group consisting of K, Na, Mn, Mg and Cu,
wherein an inducer (or stimulator) of laccase production, such as cupper or manganese ions, or a small-molecule aromatic compound (e.g. tryptophan, tyrosine, vanillin, veratryl alcohol, guaiacol, cinnamic acid, xylidine and lignin), is added to the fermentation medium (to improve laccase production by the microorganism),
wherein the fermentation is conducted aerobically, and
wherein the bioreactor is kept in agitation at between 100 rpm and 220 rpm, preferably between 120 rpm and 200 rpm.

(2) The method according to (1), wherein the Coriolopsis gallica strain is Coriolopsis gallica 1184.

(3) The method according to (1) or (2), wherein the monosaccharide, the disaccharide, the oligosaccharide and/or the polysaccharide is not bound to a non-carbohydrate substance such as lignin.

(4) The method according to any of (1) to (3), wherein the fermentation medium comprises at least 20 g/L or at least 30 g/L, preferably at least 40 g/L, such as between 40 g/L and 60 g/L (e.g. about 50 g/L) of the monosaccharide, the disaccharide, the oligosaccharide and/or the polysaccharide carbon source.

(5) The method according to any one of (1) to (4), wherein the nitrogen source is bacto™peptone and wherein the carbon source is a monosaccharide, preferably glucose.

(6) The method according to any one of (1) to (5), wherein the fermentation medium comprises 10 to 25 g/L, preferably 17 g/L, bacto™peptone; 40 to 60 g/L, preferably 50 g/L, glucose; 0.5 to 5.0 g/L, preferably 2.5 g/L, KH₂PO₄; 0.01 to 0.10 g/L, preferably 0.05 g/L, MnSO₄xH₂O; 0.1 to 1.0 g/L, preferably 0.5 g/L, MgSO₄x7H₂O; and 0.005 to 0.05 g/L, preferably 0.02 g/L, CuSO₄x5H₂O.

(7) The method according to any one of (1) to (6), wherein the inducer of laccase production is vanillin.

(8) The method according to any one of (1) to (7), wherein between 100 μM and 500 μM, preferably 200 μM, vanillin is added to the fermentation medium.

(9) The method according to any one of (1) to (8), wherein agitation speed is increased with fungal biomass during fermentation.

(10) The method according to any one of (1) to (9), wherein the temperature during the fermentation is in the range from 25° C. to 32° C., preferably in the range from 27° C. to 30° C., more preferably about 30° C.

(11) The method according to any one (1) to (10), wherein the level of dissolved oxygen is at least 25% of the saturation amount, preferably between 25% and 35% of the saturation amount.

(12) The method according to any one of (1) to (11), wherein the pH during the fermentation is in the range from 3.0 to 7.0, preferably from 3.5 to 6.0, more preferably from 3.8 to 5.5.

(13) The method according to any one of (1) to (12), wherein the fermentation period is less than 10 days, preferably between 6 and 8 days, more preferably 7 days.

(14) The method according to any one of (1) to (13), further comprising the steps of growing said Coriolopsis gallica fungal strain in an inoculum medium to obtain an inoculum, and adding said inoculum to the fermentation medium.

(15) The method according to (14), wherein the inoculum medium comprises glucose, peptone, yeast extract, and minerals.

(16) The method according to any one of (1) to (15), further comprising the step of recovering the laccase from the fermentation medium.

(17) The method according to claim (16), wherein said recovery comprises biomass separation and optionally one or more concentration steps resulting in a concentrated laccase solution.

(18) The method according to (17), wherein the concentration steps comprise a microfiltration step, an ultrafiltration step and a diafiltration step.

(19) The method according to (17) or (18), wherein the concentrated laccase solution is lyophilized or atomized.

(20) A laccase enzyme product obtainable by the method according to any one of (1) to (19).

(21) The laccase enzyme product according to (20), characterized in that that the laccase enzyme is a thermostable enzyme having a maximum activity at 72° C. and wherein the laccase enzyme retains at least 20% activity relative to the maximum activity after 20 min incubation at a temperature between 80° C. and 100° C.

(22) The laccase enzyme product according to (20) or (21), wherein the laccase enzyme retains at least 50% activity relative to the maximum activity after 20 min incubation at a temperature of 80° C.

(23) The laccase enzyme product according to any one of (20) to (22), wherein the laccase enzyme retains at least 30% activity relative to the maximum activity after 20 min incubation at 91° C.

(24) The laccase enzyme product according to any one of (21) to (23), wherein the laccase enzyme retains at least 20% activity relative to the maximum activity after 20 min incubation at 100° C.

(25) The laccase enzyme product according to any one of (21) to (24), wherein the laccase enzyme retains at least 10% activity relative to the maximum activity after 3 min incubation at 120° C.

(26) The laccase enzyme product according to any one of (21) to (25), wherein the laccase enzyme is N-glycosylated.

(27) The laccase enzyme product according to any one of (21) to (26), wherein the laccase enzyme is N-glycosylated in at least one, preferably at least two, glycosylation site selected from the group consisting of: glycosylation site FOLNVIDNMTNHTMLK (SEQ ID NO:1) and glycosylation site DVVSTGSPGDNVTIR (SEQ ID NO:2).

(28) The laccase enzyme product according to (26) or (27), wherein the laccase enzyme has a high mannose type glycosylation (i.e. the laccase enzyme glycoprotein comprises glycans that consist of two N-acetylglucosamines and five to nine mannose residues).

(29) The laccase enzyme product according to any one of (26) to (28), wherein the laccase enzyme has a level of N-glycosylation of between 10% and 20%, preferably of about 15%.

The herein disclosed aspects and embodiments of the invention are further supported by the following non-limiting examples.

### EXAMPLES

### Example 1: Production of C. gallica Laccase

### Materials and Methods

### Organism and Inoculum Preparation

Coriolopsis gallica 1184 was obtained from the culture collection of Laboratoire de Microbiologie Appliquée, Université libre de Bruxelles. Coriolopsis gallica 1184 was maintained on a 2% (w/v) malt agar plate at 4° C.

The inoculum was prepared by growing the strain on a rotary shaker at 120 rpm and 30° C. in 500 ml flasks containing 200 ml of the following defined medium (g/L): glucose 10; KH₂PO₄ 0.8; Na₂HPO₄ 0.2; MgSO₄x7H₂O 0.5; yeast extract 2.0; bacto™peptone 2.0. Each flask was inoculated with 5 malt agar-plugs (6 mm diameter) coming from solid culture on a Petri dish. After 4 days of cultivation, mycelial pellets were harvested and homogenized with a laboratory blender, three times 20 s with 1 min interval. Mycelial homogenates (200 ml) were used to inoculate a flask containing 2 L of the above medium. After 5 days of cultivation all fungal biomass (concentrated in 0.5 L) was inoculated to a bio-reactor (BIOSTAT® Plus, 10 L working volume) containing the same medium for the final step of C. gallica inoculum preparation. Growth was carried out at 30° C. during 3 days. The minimum level of dissolved oxygen was held at 25% by controlling the impeller speed 120 rpm.

### Production of Laccase at 200 L Scale

Large-scale production of laccase was performed in Techfors 200 L (TOPOCCASION) stirred bioreactor with a 200-litre working volume, containing the following defined medium (g/L): bacto™peptone 17; glucose 50; KH₂PO₄ 2.5; MnSO₄xH₂O 0.05; MgSO₄x7H₂O 0.5; CuSO₄x5H₂O 0.02. Vanillin 200 UM was added to the medium as an inducer of laccase production. The medium was sterilized for 30 min at 121° C. The prepared bioreactor was inoculated by 10 L inoculum. The temperature was controlled at 30° C. and the initial pH was 6.0. Addition of an antifoam Y-30 Emulsion (Sigma) reagent was defined as 1 ml per 24 h. Dissolved oxygen was fixed at 25% of saturation by variation of the aeration rate (1-40 m³ h) and agitation rate 120 rpm.

### Downstream Processes: Concentration of Extracellular Laccase and Dehydratation

The culture liquid was first separated from solids by filtration by metal basket 100 μm porosity. Microfiltration was per-formed as frontal-filtration using filter 0.8 μm porosity. Cross flow-ultrafiltration was realized with a polyethersulfone (PES) membrane (0.5 m², 10 kDa) on a Pellicon holder (Millipore) at a pressure of 1.5 bar. The concentrated culture liquid (15 L) was diafiltrated against a 10 volume distillate ultrapure water (pH 6.3). Before storage 3 L of concentrate was lyophilized by Christ epsilon 2-6d Iscplus (Lyophilisater). The temperature of laccase lyophilization was −42° C. for 4 days. At the same time, 3 L of concentrate was atomized in NIRO Atomosater. Before atomization 180 g of Maltodextrose was added in 3 L concentrate as laccase matrix during atomization. The time of laccase atomization was less the 1 min for each enzyme particle at 90° C. Before packaging, lyophilized and atomized laccase activity was assayed. As lyophilized also atomized laccases were packaged into the polyethylene packets with low air concentration and humidity.

### Biomass Assay

The sample volume content of the fermentation broth (500 mL) was centrifugated 4500 rpm to separate biomass from the culture liquid. The separated biomass was dried to a constant weight in an oven (70° C.). The dried biomass was weighed using an analytical balance. The biomass samples were analysed every day during the fermentation.

### Glucose Concentration Assay

Glucose concentration was determined by HPLC (Alliance e 2695, Waters), using a Shodex sugar SH-G guard column, a Shodex SH-1011 column 8 mm ID×300 mm (Waters), mobile phase H2SO4 0.01 N; flow rate 0.8 ml/min; at 40° C.; with an injected volume of 25 μl; refractometer detector 410 (Waters). Empower 2 (Waters) was used for data acquisition.

### Kinetic Modelling

The measured batch fermentation profiles of biomass concentration (X), glucose concentration(S) and laccase activity (P) were simulated using unstructured kinetic models. The fermentation kinetic parameters were estimated using nonlinear regression to fit the models to the measured data. Levenberg-Marquardt (LM) algorithm based on iterative solution method was used in obtaining the solutions to the model equations.

### Model of Fungal Growth

The dry biomass concentration was modeled using the logistic equation which describes as follows:

$$\frac{dX}{dt} = {μmax}^{X} ( 1 - \frac{X}{X max} ) \qquad ( 1 )$$

where dX/dt is the rate of biomass production (g L⁻¹ days⁻¹), μmax is the maximum specific growth rate (days⁻¹), X is the biomass concentration (g L⁻¹) and Xmax is the model predicted maximum biomass concentration for the fermentation (g L⁻¹). The integrated form of Eq. (1) is the following:

$$X = \frac{{X}_{0} exp ( {μ}_{{max}^{t}} )}{1 - ( {X}_{0} / {X}_{max} ) ( 1 - exp ( {μ}_{{max}^{t}} )} \qquad ( 2 )$$

where X₀ is the initial biomass concentration (g L⁻¹) and t is time (days).

### Model of Laccase Production

The production of laccase was modeled using the Luedeking-Piret (Luedeking and Piret, 1959) equation which describes as follows:

$$\frac{dP}{dt} = α ( \frac{dX}{dt} ) + β X \qquad ( 3 )$$

where dP/dt is the rate of laccase production (U L⁻¹ days⁻¹), dx/dt is the rate of biomass production (g L⁻¹ days⁻¹), X is the biomass concentration (g L⁻¹), a is a growth associated constant (U g⁻¹) and β is a nongrowth associated constant (U g⁻¹ days⁻¹). The values of α and β depend on the fermentation conditions. A substitution of the Eqs. (1) and (2) in (3) results in the following relationship:

$$\frac{dP}{dt} = α [ {μ}_{{max}^{X}} ( 1 - \frac{X}{{X}_{max}} ) ] + β [ \frac{{X}_{0} exp ( {μ}_{{max}^{t}} )}{1 - ( {X}_{0} / {X}_{max} ) ( 1 - exp ( {μ}_{{max}^{t}} ) )} ] \qquad ( 4 )$$

Equation (4) can be integrated using the initial condition t=0, X=X₀ and P=P₀, to produce the following equation:

$$P - {P}_{0} = [ \frac{{x}_{0} ( exp ( {μ}_{{max}^{t}} ) )}{1 - ( {X}_{0} / {X}_{max} )} - {X}_{0} ] + β [ ( \frac{{X}_{max}}{{μ}_{max}} ) In ( 1 - ( \frac{{X}_{0}}{{X}_{max}} ) ( 1 - exp ( {μ}_{{max}^{t}} ) ) ] \qquad ( 5 )$$

where P is the laccase concentration (U L⁻¹), P₀ is the initial laccase concentration (U L⁻¹) and t is time (days).

### Glucose Consumption Model

The glucose concentration was modelled as follows:

$$- \frac{dS}{dt} = \frac{1}{{Y}_{G}} ( \frac{dX}{dt} ) + {m}_{s} X \qquad ( 6 )$$

where dS/dt is the rate of glucose consumption (g L⁻¹ days⁻¹), dx/dt is the rate of biomass production (g L⁻¹ days⁻¹), X is the biomass concentration (g L⁻¹), Y is the model predicted biomass yield coefficient on glucose (g g⁻¹) and m_{S} is the cell maintenance coefficient on glucose (g g⁻¹ days⁻¹). A substitution of Eqs. (1) and (2) in (6) produced the following equation:

$$- \frac{dy}{dx} = \frac{1}{{Y}_{G}} [ {μ}_{max} X ( 1 - \frac{X}{{X}_{max}} ) ] + {m}_{s} [ \frac{{x}_{0} exp ( {μ}_{{max}^{t}} )}{1 - ( {X}_{0} / {X}_{max} ) ( 1 - exp ( {μ}_{{max}^{t}} ) )} ] \qquad ( 7 )$$

Integration of Eq. (7) for the initial condition, t=0, X=X₀ and S=S₀, led to the following relationship:

$$S - {S}_{0} = - \frac{1}{{Y}_{G}} [ \frac{{X}_{0} exp ( {μ}_{{max}^{t}} ) )}{1 - ( {X}_{0} / {X}_{max} ) ( 1 - exp ( {μ}_{{max}^{t}} ) )} - {X}_{0} ] - {m}_{s} [ ( \frac{{X}_{max}}{{μ}_{max}} ) In ( 1 - ( \frac{{X}_{0}}{{X}_{max}} ) ( exp ( {μ}_{{max}^{t}} ) ) ) ] \qquad ( 8 )$$

where S is glucose concentration (g L⁻¹), S₀ is the initial glucose concentration (g L⁻¹) and t is time (days).

### Yield Factors and Productivity

The volumetric productivities of biomass (r_{X}, gL⁻¹ days 1) and laccase (r_{P}, UL⁻¹ days⁻¹) were calculated using the following equations:

$${r}_{X} = \frac{1}{{V}_{f}} [ \frac{{X}_{f} {V}_{f} - {X}_{0} {V}_{0}}{{t}_{f} - {t}_{0}} ] \qquad ( 9 )$$

$${r}_{P} = \frac{1}{{V}_{f}} [ \frac{{P}_{f} {V}_{f} - {P}_{0} {V}_{0}}{{t}_{f} - {t}_{0}} ] \qquad ( 10 )$$

where X_{f} is the final biomass concentration, V_{f} is the final fermentation volume in the fermenter, tris the time at the end of the fermentation and P_{f} is the final concentration of laccase. Yield factors were calculated as follows:

$${Y}_{XS} = \frac{{X}_{f} - {X}_{0}}{{S}_{0} - {S}_{f}} \qquad ( 11 )$$

$${Y}_{PX} = \frac{{P}_{f} - {P}_{0}}{{X}_{f} - {X}_{0}} \qquad ( 12 )$$

$${Y}_{ps} = \frac{{P}_{f} - {P}_{0}}{{S}_{0} - {S}_{f}} \qquad ( 13 )$$

Where Y_{XS} is the biomass yield on substrate (glucose), S₀ is the initial glucose concentration, S_{f} is the final glucose concentration in the fermenter, Y_{PX} is the biomass specific yield of laccase and Y_{PS} is the laccase yield on substrate. Variables in Eqs. (9-13) were measured from the experiments.

### Laccase Activity Assay

Laccase activity was determined by monitoring the A420 change related to the rate of oxidation of 1 μmol 2,2-azinobis-[3-ethylthiazoline-6-sulfonate] (ABTS) in 100 μM Na-acetate buffer (pH 4.5). Assays were performed in a 1 ml spectrophotometric cuvette at room temperature with adequately diluted culture liquid. One unit of laccase activity was defined as the amount of enzyme, which leads to the oxidation of 1 μmol of ABTS per minute.

### Results

TABLE 1
Experimental data of C. gallica fermentation in 200 L bioreactor

| | Biomass | Laccase activity | red. saccharides |
|---|---|---|---|
| Day | [g L⁻¹] | [U L⁻¹] | [g L⁻¹] |
| 0 | 0.14 | 209 | 44.90 |
| 0.8 | 0.66 | 278 | 41.98 |
| 1.7 | 1.27 | 390 | 41.51 |
| 2.8 | 1.57 | 1209 | 41.87 |
| 3.8 | 1.74 | 1445 | 38.78 |
| 5.0 | 2.71 | 5755 | 37.77 |
| 6.6 | 2.75 | 15846 | 38.29 |
| 7.6 | 3.65 | 20016 | 36.96 |
| 8.6 | 3.71 | 25600 | 35.57 |
| 9.6 | 4.14 | 30580 | 31.61 |

Glucose was the main energy source for C. gallica biomass production and laccase expression in pilot scale. The initial concentration of glucose was 44.9 g L⁻¹ (Table 1). Comparatively low glucose consumption was observed in the pilot study compared to the lab scale study (Songulashvili et al. 2016): only 13 g/L glucose was used by C. gallica during the fermentation process (Table 1), whereas the same fungi used 45 g/L glucose at 50 L bioreactor scale in the lab scale study. Without wishing to be bound by theory, this may be due to inappropriate mixing which does not allow same concentration of biomass and glucose in all part of bioreactor. In accordance with low consumption of carbon source (glucose), the production of the biomass was low (4.14 g/L) (Table 1) compared to 50 L scale fungal fermentation (Songulashvili et al. 2016).

The initial laccase concentration was 209 U L⁻¹ after inoculation of C. gallica biomass from the inoculum preparation bioreactor (10 L) into the laccase production reactor (200 L).

At the first phase of fermentation C. gallica fungal inoculum started an intensive “growth phase” wherein approximately 40% of total biomass was produced in three days, and later laccase expression entered an intensive phase. After production of a critical biomass for enzyme expression, C. gallica produced 5000 U L⁻¹ of laccase each day of the fermentation (Table 1). Finally, C. gallica produced 30580 U L⁻¹ laccase at pilot scale fermentation.

The kinetic parameters of glucose consumption, biomass growth and laccase expression are shown in Table 2.

TABLE 2
Kinetic parameters for C. gallica fermentation in 200 L bioreactor.

| Parameter | Estimate | Approximate standard error* |
|---|---|---|
| X₀ (g L⁻¹) | 0.5411 | 0.1427 |
| μₘₐₓ (days⁻¹) | 0.4379 | 0.0954 |
| Xₘₐₓ (g L⁻¹) | 4.3637 | 0.5081 |
| α (U g⁻¹) | −1554.4 | 904.9 |
| β (U g⁻¹ days⁻¹) | 1272.9 | 248.4 |
| Y_{G} (g g⁻¹) | 1.0079 | 1.5525 |
| mₛ (g g⁻¹ days⁻¹) | 0.286 | 0.2467 |

*Approximate standard error characterizes the precision of model's parameters estimation.

The initial biomass concentration (X₀), the model predicted maximum biomass concentration (Xₘₐₓ) and the maximum specific growth rate (μₘₐₓ) in Table 2 were calculated by fitting the model (Eq. 2) to the measured fermentation profiles. C. gallica maximum specific growth rate (μₘₐₓ) was 0.4379 days⁻¹. Luedeking-Piret model (Eq. 4) was fitted to the measured data of extracellular laccase production to determine the values of the kinetic parameters a and B. These parameters are shown in Table 2. The biomass yield on glucose (Y_{G}) and the cell maintenance coefficient (m_{S}) were estimated by fitting Eq. (8) to the measured profile of glucose consumption. The estimated best fit values of these parameters are shown in Table 2.

TABLE 3
Output of laccase yield on different stage
downstream of C. gallica culture liquid.

| | | | Laccase | |
|---|---|---|---|---|
| | | Laccase | absolute | Laccase |
| | Volume | activity | activity | distribution |
| Downstream steps | [L] | [U L⁻¹] | [U] | [%] |
| Supernatant and | 190 | 30580 ± 380 | 5810200 | 100 |
| biomass separation | | | | |
| (100 μm) | | | | |
| Micro-filtration | 150 | 30580 ± 410 | 4587000 | 100 |
| (0.8 μm) | | | | |
| Ultra-filtration | 15 | 302742 ± 3800 | 4541130 | 99 |
| (10 kDa) | | | | |
| Dia-filtration × 15 | 15 | 302742 ± 210 | 4541130 | 99 |

After fungal fermentation supernatant was separated form fungal biomass and colloidal particles by gravity at 100 μm porosity metal basket. The total volume of fungal culture liquid was 190 L with 30580 U L⁻¹ laccase activity. Our microfiltration separation process was based on 0.8 μm pore-sized membranes. In our study, 150 L culture liquid was micro-filtrated. The concentration of laccase was 100% identical compared to culture liquid before microfiltration (Table 3). The partial purification concentration microfiltrated culture liquid was done by ultrafiltration pore sized 10 kDa. The culture liquid was concentrated ten times with 99% similar activity compared to microfiltration. Finally, 15 L of culture liquid with laccase concentration 302742 U L⁻¹ was dia-filtrated to maximally eliminate sugars and metabolized compounds.

For increasing stability of laccase during the storage, a part of concentrated culture liquid was lyophilized and another part was atomized. The total mass balance of fungal laccase production was 240 g having a specific activity of 18 700 U mg⁻¹ of protein. Samples were vacuum packed in polyethylene bags for storage.

### Example 2: Thermostability of C. gallica Laccase

### Materials and Methods

0.010 g powder of lyophilized Coriolopsis gallica laccase obtained according to example 1 was used for each temperature tested (72, 80, 91, 100 and 120° C.). Laccase samples were heated in a PCR machine. Laccase thermal resistance assay was done after heating for 3, 10, 20 min at each temperature tested. Laccase activity was tested as described in example 1. The test was done in triplicate for each temperature. The thermostability is expressed as the laccase activity after heat treatment relative to the laccase activity in control (not heated).

### Results

TABLE 4
C. gallica laccase thermostability.

| T (° C.) | 3 min | 10 min | 20 min |
|---|---|---|---|
| 120 | 10% | trace | ND |
| 100 | 61.5% | 35.3% | 20.7% |
| 91 | 70.6% | 46.1% | 30.7% |
| 80 | 100% | 67.6% | 52.3% |
| 72 | 100% | 100% | 100% |

### Example 3: C. gallica Laccase Identification by Liquid Chromatography-Electrospray Ionization-Tandem Mass Spectrometry (LC-ESI-MS/MS)

### Materials and Methods

The analysis was performed on an LC (nano Ultimate 3000-Dionex)-ESI-ion trap (AmaZon Speed ETD-Bruker Daltonics), in positive ion mode. Laccase obtained according to Example 1 was purified substantially as described in Songulashvili et al. (2016). The protein content of the purified sample was determined using RCDC kit form Biorad according to the manufacturer's instructions. The obtained concentration was 10 mg/mL. The sample was reduced, alkylated, and ultrafiltrated using Amicon (Millipore) with membrane cut-off of 3 kDa, to remove salts and placed in ammonium bicarbonate. Sample was then digested using trypsin. After the digestion was stopped, one half of the sample was treated with PNGase F and digested further using trypsin (experiment 14/1994-25). The other half of the sample was directly digested further using trypsin (experiment 14/1996-26). The protein digests were independently analyzed by LC-ESI-MS/MS. Spectra were treated using Data analysis vs 4.0 (Bruker). Databases searches were performed using the Mascot server vs 2.2.04 and Protein Scape vs. 3.0 (Bruker), on NCBI including Fungi taxonomies. For experiment 14/1996-25, Carbamidomethyl of Cysteines (C, resulting from alkylation before digestion), Deamidation of Asparagine and Glutamine (N, Q) and Oxidation of Methionine (M) were set as variable modifications. For experiment 14/1996-26, Carbamidomethyl of Cysteines (C, resulting from alkylation before digestion) and Oxidation of Methionine (M) were set as variable modifications.

A Fetuin Quality Control sample was digested in parallel with the sample (one half with PNGase F treatment and the other half without deglycosylation) and database search was performed on SwissProt including all taxonomies, to monitor the whole process.

### Results

### Glycosylation Analysis on Sample 14/1994

Based on the results obtained after PNGase F deglycosylation, two potential N-glycopeptides (containing the consensus sequence NXS/NXT with X amino acid except P, and being deamidated due to the PNGase F) were found: FOLNVIDNMTNHTMLK (SEQ ID NO:1) and DVVSTGSPGDNVTIR (SEQ ID NO: 2).

The MS/MS and MS spectra obtained for the sample experiment 14/1994-26 were analyzed in order to find glycopeptides. This was done by searching for the reporter ion at m/z=366 (that is usually produced by collision induced dissociation of glycopeptides). Two interesting chromatographic regions were determined at RT=20-21 min and 26.5-27 min.

The MS spectra in which these two peaks were found were analyzed and several peaks separated by +/−54 m/z were found. Taking into account that these peaks are all 3+, the mass difference between those peaks is 162 Da, corresponding to Hexose additions. These results indicate that the glycans are from the “High Mannose” type. For the first glycopeptide (sequence DVVSTGSPGDNVTIR (SEQ ID NO: 2)), at least four glycoforms containing from 5 to 8 mannoses were observed.

In order to determine if one site is preferentially occupied, Electron Transfer Dissociation experiments were performed. This activation method allows fragmenting the peptidic part of the glycopeptide, giving information about the sequence that carries the glycan. The ion at m/z=910.6 (4+) was therefore subjected to ETD. The results show that a higher sequence coverage was obtained when the glycan is positioned on the second possible site, indicating that this site is more likely occupied than the other one.

14/1994-26 (with deamidation as variable modification) provided significant identifications:

| Row | Accession | Protein | MW [kDa] | pi | Scores | #Peptides | SC [%] | RMS90 [ppm] |
|---|---|---|---|---|---|---|---|---|
| 1 | gi [symbol] 58324 | laccase [basidiomycate PM1] | 55.5 | 4.8 | 941.5 (M: 941.5) | 21 | 24.4 | 2 [symbol] .03 |
| 2 | gi [symbol] 171684547 | hypothetical protein | 66.0 | 9.7 | 96.8 (M: 96.8) | 2 | 2.0 | 3 [symbol] .09 |
| | | [Podospora anserina [symbol] ] | | | | | | |

[symbol]

indicates data missing or illegible when filed

14/1994-26 provided significant identifications:

| Row | Accession | Protein | MW [kDa] | pi | Scores | #Peptides | SC [%] | RMS90 [ppm] |
|---|---|---|---|---|---|---|---|---|
| 1 | gi [symbol] 58324 | laccase [basidiomycate PM1] | 55.5 | 4.8 | 489.9 (M: 489.9) | 6 | 14.9 | 47.37 |
| 2 | gi [symbol] 12484399 | laccase [Coriolopsis gallica] | 56.5 | 5.7 | 176.2 (M: 176.2) | 2 | 7.5 | 91.87 |

[symbol]

indicates data missing or illegible when filed

### Example 4: Production and Characterization of Laccases from Different C. gallica Strains

C. gallica laccase was produced according to example 1 using 2 other C. gallica strains: C. gallica CBS 547.50 and C. gallica CBS 576.88 available at the CBS Filamentous Fungi Collection (Utrecht, The Netherlands). Thermostability of the produced laccases was tested as described in example 2.

TABLE 5
C. gallica strain CBS 547.50 laccase thermostability.

| T (° C.) | 3 min | 10 min | 20 min |
|---|---|---|---|
| 120 | 0 | 0 | 0 |
| 100 | 0 | 0 | 0 |
| 91 | 34.6 | 29.9 | 23.0 |
| 80 | 100 | 100 | 100 |
| 72 | 100 | 100 | 100 |

TABLE 6
C. gallica strain CBS 576.88 laccase thermostability.

| T (° C.) | 3 min | 10 min | 20 min |
|---|---|---|---|
| 120 | 1.0 | 0 | 0 |
| 100 | 0.9 | 0 | 0 |
| 91 | 61.3 | 30.7 | 17.5 |
| 80 | 100 | 86.9 | 87.7 |
| 72 | 100 | 100 | 96.1 |

1. A lyophilized laccase enzyme of a Coriolopsis gallica fungal strain, wherein the laccase enzyme has maximum activity at 72° C. and wherein the laccase enzyme retains at least 50% activity relative to the maximum activity after 20 min incubation at a temperature of 80° C.

2. The laccase enzyme of claim 1, wherein the laccase enzyme retains at least 15% activity relative to the maximum activity after 20 min incubation at a temperature between 80° C. and 91° C.

3. The laccase enzyme of claim 1, wherein the laccase enzyme retains at least 20% activity relative to the maximum activity after 20 min incubation at a temperature between 80° C. and 100° C.

4. The laccase enzyme of claim 1, wherein the laccase enzyme retains at least 30% activity relative to the maximum activity after 20 min incubation at 91° C.

5. The laccase enzyme of claim 1, wherein the laccase enzyme retains at least 10% activity relative to the maximum activity after 3 min incubation at 120° C.

6. The laccase enzyme of claim 1, wherein the laccase enzyme is N-glycosylated in at least a glycosylation site of SEQ ID NO:1 (FQLNVIDNMTNHTMLK) and/or a glycosylation site of SEQ ID NO:2 (DVVSTGSPGDNVTIR).

7. The laccase enzyme of claim 6, wherein the laccase enzyme has a level of N-glycosylation of between 10% and 20%.

8. A method for the production of the laccase enzyme of claim 1, the method comprising:
fermenting, via submerged fermentation, a Coriolopsis gallica fungal strain in a fermentation medium in an agitated tank bioreactor of at least 200 L, for a fermentation period,
wherein the fermentation medium comprises at least 20 g/L of a carbon source selected from a monosaccharide, a disaccharide, an oligosaccharide, a polysaccharide or any combination thereof, wherein the monosaccharide, the disaccharide, the oligosaccharide and/or the polysaccharide is not bound to lignin; a nitrogen source; and minerals,
wherein an inducer of laccase production is added to the fermentation medium,
wherein the fermentation is conducted aerobically, and
wherein the bioreactor is kept in agitation at between 100 rpm and 220 rpm.

9. The method according to claim 8, wherein the carbon source is a monosaccharide, preferably glucose, and wherein the nitrogen source is peptone.

10. The method according to claim 8, wherein between 100 μM and 500 μM vanillin is added to the fermentation medium.

11. The method according to claim 8, wherein the temperature during the fermentation is from 25° C. to 32° C.

12. The method according to claim 8, wherein the level of dissolved oxygen is at least 25% of the saturation amount.

13. The method according to claim 8, wherein the fermentation period is less than 10 days.

14. The method according to claim 8, further comprising the steps of growing the Coriolopsis gallica fungal strain in an inoculum medium to obtain an inoculum, and adding the inoculum to the fermentation medium.

15. The method according to claim 8, further comprising the step of recovering the laccase from the fermentation medium.

16. The method according to claim 15, wherein the recovery comprises biomass separation and optionally one or more concentration steps resulting in a concentrated laccase solution.

17. The method according to claim 16, wherein the concentration steps comprise a microfiltration step, an ultrafiltration step and a diafiltration step.

18. The method according to claim 16, wherein the concentrated laccase solution is lyophilized or atomized.
